(19) United States
(12) Patent Application Publication
(10) Pub. No.: US 2021/0251964 A1
(43) Pub. Date: Aug. 19, 2021

(54) POLYMORPHS OF 5-({[2-AMINO-3-(4-CARBAMOYL-2,6-DIMETHYL-PHENYL)-PROPIONYL]-[1-(4-PHENYL-1H-IMIDAZOL-2-YL)-ETHYL]-AMINO}-METHYL)-2-METHOXY-BENZOIC ACID

(71) Applicant: Allergan Holdings Unlimited Company, Dublin (IE)

(72) Inventors: Graham Lawton, Madison, NJ (US); Lia Faherty, Parsippany, NJ (US); Scott Trzaska, Raritan, NJ (US)

(21) Appl. No.: 17/269,873

(22) PCT Filed: Aug. 19, 2019
(86) PCT No.: PCT/IB2019/056988

§ 371 Date: Feb. 19, 2021

Related U.S. Application Data
(60) Provisional application No. 62/719,756, filed on Aug. 20, 2018.

(51) Int. Cl. A61K 31/417; A61K 9/00

(52) U.S. Cl. CPC A61K 31/417; A61K 9/0053

(57) ABSTRACT

The present invention relates to novel crystalline Forms D, E and F of 5-({[2-amino-3-(4-carbamoyl-2,6-dimethyl-phenyl)-propionyl]-[1-(4-phenyl-1H-imidazol-2-yl)-ethyl]-amino}-methyl)-2-methoxy-benzoic acid, methods of preparing the same and their pharmaceutical compositions for use in treating opioid receptor mediated diseases.

### FIELD OF THE INVENTION

The present invention relates to novel crystalline forms of 5-({[2-amino-3-(4-carbamoyl-2,6-dimethyl-phenyl)-propionyl]-[1-(4-phenyl-1h-imidazol-2-yl)-ethyl]-amino}-methyl)-2-methoxy-benzoic acid, methods of making them and uses thereof in the preparation or production of pharmaceutical drug dosage forms.

### BACKGROUND OF THE INVENTION

The delivery of an active pharmaceutical ingredient (“API”) to a patient requires more than simply the identification of a molecule and its methods of use. An API must be formulated for delivery to a patient and such formulation (in addition to the API activity) is evaluated by regulatory agencies such as the US Food and Drug Administration (FDA) and the European Medicines Agency (EMEA). The API's formulation affects, among others, delivery profile, stability, consistency, and manufacturing controls. An important factor in determining the properties of a formulation is the form of the API. APIs have been known to exist as amorphous forms, crystalline forms, polymorphs, hydrates and solvates. While one API may be known to have one or multiple polymorph or solvate forms in addition to its amorphous form, another API may be known to only exist in amorphous form. The form diversity is important because each different polymorphic form, (i.e. anhydrous, crystalline solvate(s), crystalline hydrate(s) or amorphous form) may have different physicochemical properties such as stability, solubility, dissolution rate, melting temperature and hygroscopicity.

Some forms of an API can be formulated into a pharmaceutical formulation suitable for human use, while other forms lack the required properties for such uses. Even if an API can exist in more than one form suitable for formulation, different properties of an API form can affect the manufacturing process, shelf-life, route of administration, bioavailability and other important product characteristics. For example, the ability to improve or modulate stability or hygroscopicity can decrease manufacturing costs by reducing the need for humidity controlled chambers or reducing the need to package an API in humidity resistant packaging. In addition, these same changes can increase product shelf stability thereby improving product distribution possibilities and affecting cost. In another example, one polymorphic form of an API may have greater bioavailability than another form. Choosing a form that provides the higher bioavailability allows for a lower drug dose to be administered to a patient. Selecting the most stable hydrate of the drug for development of aqueous based oral suspension formulation(s) provides a better physical stability of the drug in oral suspension, micro-suspension and nano-suspension products, e.g., pediatric oral suspensions, micro-suspensions and nano-suspensions.

Further, changes to the process of making an API can result in less processing steps, higher purity and lower cost. Such advantages are important to the pharmaceutical industry.

5-({[2-Amino-3-(4-carbamoyl-2,6-dimethyl-phenyl)-propionyl]-[1-(4-phenyl-1H-imidazol-2-yl)-ethyl]-amino}-methyl)-2-methoxy-benzoic acid, also known as eluxadoline (structure shown in FIG. 10), is an opioid receptor modulator (mu receptor agonist and delta receptor antagonist) useful for treating irritable bowel syndrome, pain or other opioid receptor disorders. 5-({[2-Amino-3-(4-carbamoyl-2,6-dimethyl-phenyl)-propionyl]-[1-(4-phenyl-1H-imidazol-2-yl)-ethyl]-amino}-methyl)-2-methoxy-benzoic acid and methods of making this molecule are disclosed in US application 2005/02033143. Example 9 of US application 2005/02033143 makes the hydrochloride salt of 5-({[2-amino-3-(4-carbamoyl-2,6-dimethyl-phenyl)-propionyl]-[1-(4-phenyl-1H-imidazol-2-yl)-ethyl]-amino}-methyl)-2-methoxy-benzoic acid. US application 2009/0018179 describes a zwitterion of 5-({[2-amino-3-(4-carbamoyl-2,6-dimethyl-phenyl)-propionyl]-[1-(4-phenyl-1H-imidazol-2-yl)-ethyl]-amino}-methyl)-2-methoxy-benzoic acid and two crystalline forms α and β of this compound. Crystalline forms α and β provided improved properties over the amorphous form and can be purified at higher purity. The α and β crystals may be interchangeably referred to herein as “Form A” and “Form B” crystals, respectively. The entire contents of US application 2005/02033143 and 2009/0018179 are incorporated herein by reference. Other polymeric forms of eluxadoline are described in PCT/US2016/043678, published as WO 2017/015606.

Additional polymeric forms of 5-({[2-amino-3-(4-carbamoyl-2,6-dimethyl-phenyl)-propionyl]-[1-(4-phenyl-1H-imidazol-2-yl)-ethyl]-amino}-methyl)-2-methoxy-benzoic acid having different or improved properties are needed in the art and are described herein.

### SUMMARY OF THE INVENTION

The present invention relates to novel crystalline forms of 5-({[2-amino-3-(4-carbamoyl-2,6-dimethyl-phenyl)-propionyl]-[1-(4-phenyl-1H-imidazol-2-yl)-ethyl]-amino}-methyl)-2-methoxy-benzoic acid. The invention also provides pharmaceutical compositions comprising these novel crystalline forms. Compositions and methods of the invention are useful in the treatment or prevention of a variety of diseases including, among others, irritable bowel syndrome, pain and other opioid receptor mediated disorders.

In one embodiments, the invention relates to a novel Form D crystal of eluxadoline characterized by a powder X-ray diffraction pattern comprising the powder X-ray diffraction peaks at 2-theta values of about 8.0±0.2, 8.6±0.2, 8.9±0.2, 10.1±0.2, 11.0±0.2, 13.4±0.2, 14.7±0.2, 15.8±0.2, 17.7±0.2, 18.6±0.2, 20.2±0.2, 21.3±0.2, 22.1±0.2, 23.2±0.2, 24.2±0.2, 25.6±0.2, 26.9±0.2, 28.2±0.2, 29.2±0.2, 30.0±0.2, 31.0±0.2, 32.1±0.2, 33.4±0.2, 34.5±0.2, 36.7±0.2, and 38.2±0.2 degrees.

In another embodiment, the Form D crystal may be characterized by at least a minimum corresponding number of powder X-ray diffraction peaks at 8.0±0.2, 8.6±0.2, 8.9±0.2, 10.1±0.2, 11.0±0.2, 13.4±0.2, 14.7±0.2, 15.8±0.2, 17.7±0.2, 18.6±0.2, 20.2±0.2, 21.3±0.2, 22.1±0.2, 23.2±0.2, 24.2±0.2, 25.6±0.2, 26.9±0.2, 28.2±0.2, 29.2±0.2, 30.0±0.2, 31.0±0.2, 32.1±0.2, 33.4±0.2, 34.5±0.2, 36.7±0.2, and 38.2±0.2 degrees, wherein the minimum corresponding number is three, four, five, six, seven, eight, nine, ten or more than ten.

In another embodiments, the invention includes a novel Form E crystal of eluxadoline characterized by a powder X-ray diffraction pattern comprising the powder X-ray diffraction peaks at 2-theta values of about 2.1±0.2, 8.6±0.2, 10.3±0.2, 11.2±0.2, 11.8±0.2, 13.5±0.2, 15.3±0.2, 15.8±0.2, 17.4±0.2, 18.3±0.2, 19.4±0.2, 20.1±0.2, 21.6±0.2, 23.1±0.2, 24.5±0.2, 25.7±0.2, 27.8±0.2, 28.8±0.2, 30.2±0.2, 32.5±0.2, 33.4±0.2, 35.2±0.2 and 38.4±0.2.±0.2 degrees.

In another embodiment, the Form E crystal may be characterized by at least a minimum corresponding number of powder X-ray diffraction peaks of 2-theta values of about 2.1±0.2, 8.6±0.2, 10.3±0.2, 11.2±0.2, 11.8±0.2, 13.5±0.2, 15.3±0.2, 15.8±0.2, 17.4±0.2, 18.3±0.2, 19.4±0.2, 20.1±0.2, 21.6±0.2, 23.1±0.2, 24.5±0.2, 25.7±0.2, 27.8±0.2, 28.8±0.2, 30.2±0.2, 32.5±0.2, 33.4±0.2, 35.2±0.2 and 38.4±0.2.±0.2 degrees, wherein the minimum corresponding number is three, four, five, six, seven, eight, nine, ten or more than ten.

In another aspect, invention includes a novel Form F crystal of eluxadoline characterized by a powder X-ray diffraction pattern comprising any three or more powder X-ray diffraction peaks at 2-theta values of about 2.1±0.2, 6.4±0.2, 7.1±0.2, 7.5±0.2, 9.1±0.2, 10.1±0.2, 11.0±0.2, 11.4±0.2, 13.2±0.2, 14.5±0.2, 15.9±0.2, 18.0±0.2, 18.6±0.2, 18.9±0.2, 19.3±0.2, 20.0±0.2, 20.4±0.2, 21.5±0.2, 23.6±0.2, 24.9±0.2, 28.8±0.2. 31.6±0.2 and 32.4±0.2. degrees.

In another embodiment, the Form F crystal may be characterized by at least a minimum corresponding number of powder X-ray diffraction peaks of 2-theta values of about 2.1±0.2, 6.4±0.2, 7.1±0.2, 7.5±0.2, 9.1±0.2, 10.1±0.2, 11.0±0.2, 11.4±0.2, 13.2±0.2, 14.5±0.2, 15.9±0.2, 18.0±0.2, 18.6±0.2, 18.9±0.2, 19.3±0.2, 20.0±0.2, 20.4±0.2, 21.5±0.2, 23.6±0.2, 24.9±0.2, 28.8±0.2. 31.6±0.2 and 32.4±0.2 degrees, wherein the minimum corresponding number is three, four, five, six, seven, eight, nine, ten or more than ten.

### BRIEF DESCRIPTION OF THE DRAWINGS

FIG. 1 illustrates powder X-ray diffraction (PXRD) measurements of a representative crystalline Form D.

FIG. 2 illustrates a thermal gravimetric analysis (TGA) measurement of a representative crystalline Form D.

FIG. 3 illustrates a differential scanning calorimetry (DSC) measurement of a representative crystalline Form D.

FIG. 4 illustrates powder X-ray diffraction (PXRD) measurements of a representative crystalline Form E.

FIG. 5 illustrates a thermal gravimetric analysis (TGA) measurement of a representative crystalline Form E.

FIG. 6 illustrates a differential scanning calorimetry (DSC) measurement of a representative crystalline Form E.

FIG. 7 illustrates powder X-ray diffraction (PXRD) measurements of a representative crystalline Form F.

FIG. 8 illustrates a thermal gravimetric analysis (TGA) measurement of a representative crystalline Form F.

FIG. 9 illustrates a differential scanning calorimetry (DSC) measurement of a representative crystalline Form F.

FIG. 10 illustrates the structure of eluxadoline.

### DETAILED DESCRIPTION

The present invention includes novel crystalline forms of 5-({[2-amino-3-(4-carbamoyl-2,6-dimethyl-phenyl)-propionyl]-[1-(4-phenyl-1H-imidazol-2-yl)-ethyl]-amino}-methyl)-2-methoxy-benzoic acid.

In one aspect of the invention, the invention includes a novel Form D crystal of 5-({[2-amino-3-(4-carbamoyl-2,6-dimethyl-phenyl)-propionyl]-[1-(4-phenyl-1H-imidazol-2-yl)-ethyl]-amino}-methyl)-2-methoxy-benzoic acid. Form D may be useful for treating pain, irritable bowel syndrome, or other opioid receptor disorders in mammals, by administering to said mammal an effective amount of a Form D crystal of 5-({[2-amino-3-(4-carbamoyl-2,6-dimethyl-phenyl)-propionyl]-[1-(4-phenyl-1H-imidazol-2-yl)-ethyl]-amino}-methyl)-2-methoxy-benzoic acid.

In some embodiments, the Form D crystal may be characterized by a powder X-ray diffraction pattern comprising the powder X-ray diffraction peaks at 2-theta values of about 8.0±0.2, 8.6±0.2, 8.9±0.2, 10.1±0.2, 11.0±0.2, 13.4±0.2, 14.7±0.2, 15.8±0.2, 17.7±0.2, 18.6±0.2, 20.2±0.2, 21.3±0.2, 22.1±0.2, 23.2±0.2, 24.2±0.2, 25.6±0.2, 26.9±0.2, 28.2±0.2, 29.2±0.2, 30.0±0.2, 31.0±0.2, 32.1±0.2, 33.4±0.2, 34.5±0.2, 36.7±0.2, and 38.2±0.2 degrees.

In another embodiment, the Form D crystal may be characterized by at least a minimum corresponding number of powder X-ray diffraction peaks at 2-theta values of about 8.0±0.2, 8.6±0.2, 8.9±0.2, 10.1±0.2, 11.0±0.2, 13.4±0.2, 14.7±0.2, 15.8±0.2, 17.7±0.2, 18.6±0.2, 20.2±0.2, 21.3±0.2, 22.1±0.2, 23.2±0.2, 24.2±0.2, 25.6±0.2, 26.9±0.2, 28.2±0.2, 29.2±0.2, 30.0±0.2, 31.0±0.2, 32.1±0.2, 33.4±0.2, 34.5±0.2, 36.7±0.2, and 38.2±0.2 degrees, wherein the minimum corresponding number is three, four, five, six, seven, eight, nine, ten or more than ten.

In some embodiments, the Form D crystal may be characterized by a powder X-ray diffraction pattern having powder X-ray diffraction peaks at about 17.7±0.2, 20.2±0.2, and 26.9±0.2 degrees 2-theta. In some embodiments, the Form D crystal may be characterized by a powder X-ray diffraction pattern having powder X-ray diffraction peaks at about 8.9±0.2, 17.7±0.2, 20.2±0.2 and 26.9±0.2 degrees 2-theta. In some embodiments, the Form D crystal may be characterized by a powder X-ray diffraction pattern having powder X-ray diffraction peaks at about 8.6±0.2, 8.9±0.2, 17.7±0.2, 20.2±0.2 and 26.9±0.2 degrees 2-theta.

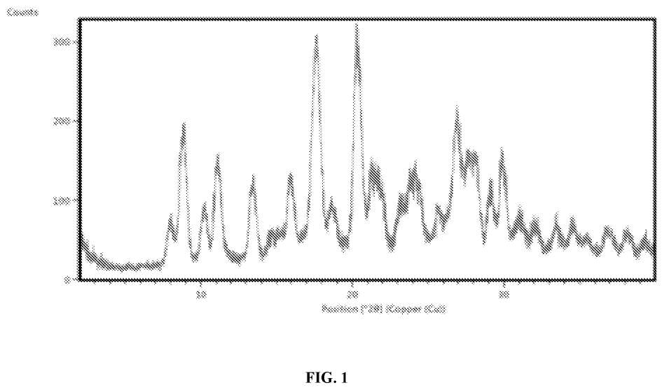

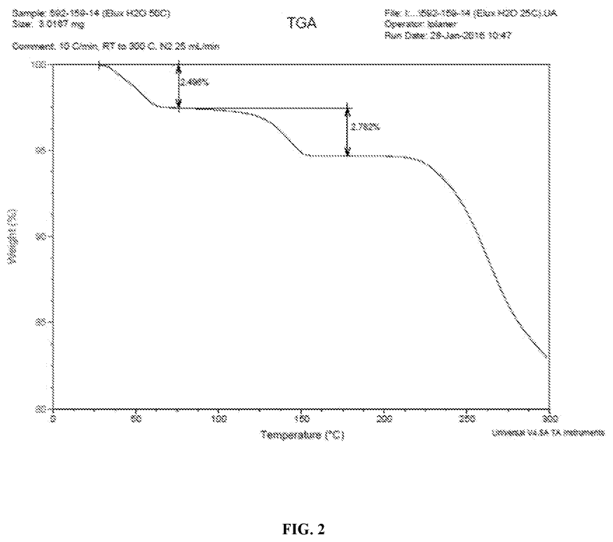

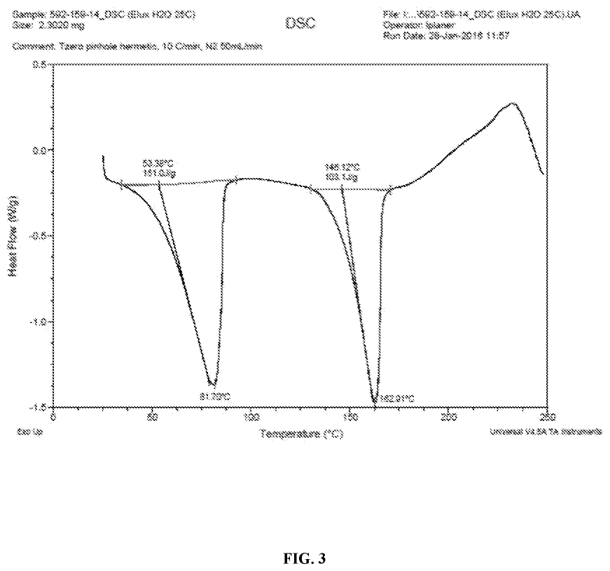

In some embodiments, the Form D crystal may be characterized by a powder X-ray diffraction pattern having powder X-ray diffraction peaks substantially as shown in Table 1. In some embodiments, the Form D crystal may be characterized by a powder X-ray diffraction pattern that may be substantially similar to the powder X-ray diffraction pattern of FIG. 1. In some embodiments, the Form D crystal may be characterized by a thermal gravimetric analysis (TGA) curve substantially similar to the TGA curve shown in FIG. 2. In some embodiments, the Form D crystal may be characterized by a differential scanning calorimetry (DSC) measurement substantially similar to the DSC in FIG. 3. In some embodiments, the Form D crystal may be substantially pure.

TABLE 1

| No. | °20 |
|---|---|
| 1 | 8.0 |
| 2 | 8.6 |
| 3 | 8.9 |
| 4 | 10.1 |
| 5 | 11.0 |
| 6 | 13.4 |
| 7 | 14.7 |
| 8 | 15.8 |
| 9 | 17.7 |
| 10 | 18.6 |
| 11 | 20.2 |
| 12 | 21.3 |
| 13 | 22.1 |
| 14 | 23.2 |
| 15 | 24.2 |
| 16 | 25.6 |
| 17 | 26.9 |
| 18 | 28.2 |
| 19 | 29.2 |
| 20 | 30.0 |
| 21 | 31.0 |
| 22 | 32.1 |
| 23 | 33.4 |
| 24 | 34.5 |
| 25 | 36.7 |
| 26 | 38.2 |

In another aspect of the invention, the present invention also includes a novel Form E crystal of 5-({[2-amino-3-(4-carbamoyl-2,6-dimethyl-phenyl)-propionyl]-[1-(4-phenyl-1H-imidazol-2-yl)-ethyl]-amino}-methyl)-2-methoxy-benzoic acid. Form E may be useful for treating pain, irritable bowel syndrome, or other opioid receptor disorders in mammals, by administering to said mammal an effective amount of a Form E crystal of 5-({[2-amino-3-(4-carbamoyl-2,6-dimethyl-phenyl)-propionyl]-[1-(4-phenyl-1H-imidazol-2-yl)-ethyl]-amino}-methyl)-2-methoxy-benzoic acid.

In some embodiments, the Form E crystal may be characterized by a powder X-ray diffraction pattern comprising the powder X-ray diffraction peaks at 2-theta values of about 2.1±0.2, 8.6±0.2, 10.3±0.2, 11.2±0.2, 11.8±0.2, 13.5±0.2, 15.3±0.2, 15.8±0.2, 17.4±0.2, 18.3±0.2, 19.4±0.2, 20.1±0.2, 21.6±0.2, 23.1±0.2, 24.5±0.2, 25.7±0.2, 27.8±0.2, 28.8±0.2, 30.2±0.2, 32.5±0.2, 33.4±0.2, 35.2±0.2 and 38.4±0.2.±0.2 degrees.

In another embodiment, the Form E crystal may be characterized by at least a minimum corresponding number of powder X-ray diffraction peaks of 2-theta values of about 2.1±0.2, 8.6±0.2, 10.3±0.2, 11.2±0.2, 11.8±0.2, 13.5±0.2, 15.3±0.2, 15.8±0.2, 17.4±0.2, 18.3±0.2, 19.4±0.2, 20.1±0.2, 21.6±0.2, 23.1±0.2, 24.5±0.2, 25.7±0.2, 27.8±0.2, 28.8±0.2, 30.2±0.2, 32.5±0.2, 33.4±0.2, 35.2±0.2 and 38.4±0.2.±0.2 degrees, wherein the minimum corresponding number is three, four, five, six, seven, eight, nine, ten or more than ten.

In some embodiments, the Form E crystal may be characterized by a powder X-ray diffraction pattern having powder X-ray diffraction peaks at about 8.6±0.2, 17.4±0.2 and 21.6±0.2 degrees 2-theta. In some embodiments, the Form E crystal may be characterized by a powder X-ray diffraction pattern having powder X-ray diffraction peaks at about 8.6±0.2, 11.8±0.2, 17.4±0.2 and 21.6±0.2 degrees 2-theta. In some embodiments, the Form E crystal may be characterized by a powder X-ray diffraction pattern having powder X-ray diffraction peaks at about 8.6±0.2, 11.8±0.2, 17.4±0.2, 21.6±0.2 and 27.8±0.2 degrees 2-theta.

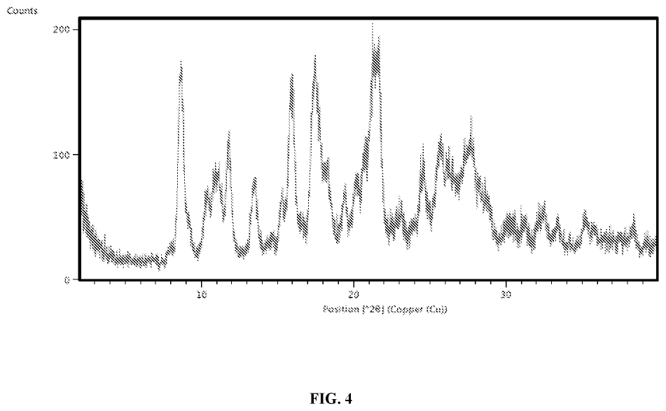

In some embodiments, the Form E crystal may be characterized by a powder X-ray diffraction pattern having powder X-ray diffraction peaks substantially as shown in Table 2. In some embodiments, the Form E crystal may be characterized by a powder X-ray diffraction pattern that may be substantially similar to the powder X-ray diffraction pattern of FIG. 4.

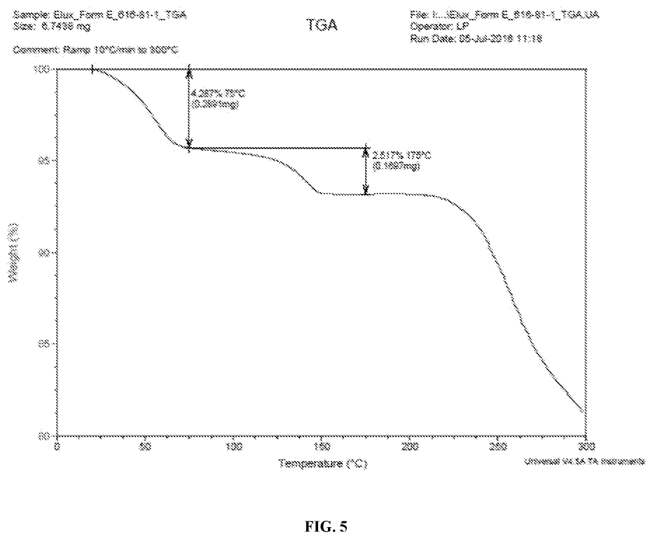

In some embodiments, the Form E crystal may be characterized by a thermal gravimetric analysis (TGA) curve substantially similar to the TGA in FIG. 5. In some embodiments, the Form E crystal may be characterized by a differential scanning calorimetry (DSC) measurement substantially similar to the DSC in FIG. 5. In some embodiments, the Form E crystal may be substantially pure.

TABLE 2

| No. | °20 |
|---|---|
| 1 | 2.1 |
| 2 | 8.6 |
| 3 | 10.3 |
| 4 | 11.2 |
| 5 | 11.8 |
| 6 | 13.5 |
| 7 | 15.3 |
| 8 | 15.8 |
| 9 | 17.4 |
| 10 | 18.3 |
| 11 | 19.4 |
| 12 | 20.1 |
| 13 | 21.6 |
| 14 | 23.1 |
| 15 | 24.5 |
| 16 | 25.7 |
| 17 | 27.8 |
| 18 | 28.8 |
| 19 | 30.2 |
| 20 | 32.5 |
| 21 | 33.4 |
| 22 | 35.2 |
| 23 | 38.4 |

In yet another aspect of the invention, the present invention also includes a novel Form F crystal of 5-({[2-amino-3-(4-carbamoyl-2,6-dimethyl-phenyl)-propionyl]-[1-(4-phenyl-1H-imidazol-2-yl)-ethyl]-amino}-methyl)-2-methoxy-benzoic acid. Form F may be useful for treating pain, irritable bowel syndrome, or other opioid receptor disorders, by administering to said mammal an effective amount of a Form F crystal of 5-({[2-amino-3-(4-carbamoyl-2,6-dimethyl-phenyl)-propionyl]-[1-(4-phenyl-1H-imidazol-2-yl)-ethyl]-amino}-methyl)-2-methoxy-benzoic acid.

In some embodiments, the Form F crystal may be characterized by a powder X-ray diffraction pattern comprising any three or more powder X-ray diffraction peaks at 2-theta values of about 2.1±0.2, 6.4±0.2, 7.1±0.2, 7.5±0.2, 9.1±0.2, 10.1±0.2, 11.0±0.2, 11.4±0.2, 13.2±0.2, 14.5±0.2, 15.9±0.2, 18.0±0.2, 18.6±0.2, 18.9±0.2, 19.3±0.2, 20.0±0.2, 20.4±0.2, 21.5±0.2, 23.6±0.2, 24.9±0.2, 28.8±0.2. 31.6±0.2 and 32.4±0.2. degrees.

In another embodiment, the Form F crystal may be characterized by at least a minimum corresponding number of powder X-ray diffraction peaks of 2-theta values of about 2.1±0.2, 6.4±0.2, 7.1±0.2, 7.5±0.2, 9.1±0.2, 10.1±0.2, 11.0±0.2, 11.4±0.2, 13.2±0.2, 14.5±0.2, 15.9±0.2, 18.0±0.2, 18.6±0.2, 18.9±0.2, 19.3±0.2, 20.0±0.2, 20.4±0.2, 21.5±0.2, 23.6±0.2, 24.9±0.2, 28.8±0.2. 31.6±0.2 and 32.4±0.2 degrees, wherein the minimum corresponding number is three, four, five, six, seven, eight, nine, ten or more than ten.

In some embodiments, the Form F crystal may be characterized by a powder X-ray diffraction pattern having powder X-ray diffraction peaks at about 6.4±0.2, 13.2±0.2 and 18.0±0.2 degrees 2-theta. In some embodiments, the Form F crystal may be characterized by a powder X-ray diffraction pattern having powder X-ray diffraction peaks at about 6.4±0.2, 13.2±0.2, 15.9±0.2, and 18.0±0.2 degrees 2-theta. In some embodiments, the Form F crystal may be characterized by a powder X-ray diffraction pattern having powder X-ray diffraction peaks at about 6.4±0.2, 10.1±0.2, 13.2±0.2, 15.9±0.2 and 18.0±0.2 degrees 2-theta.

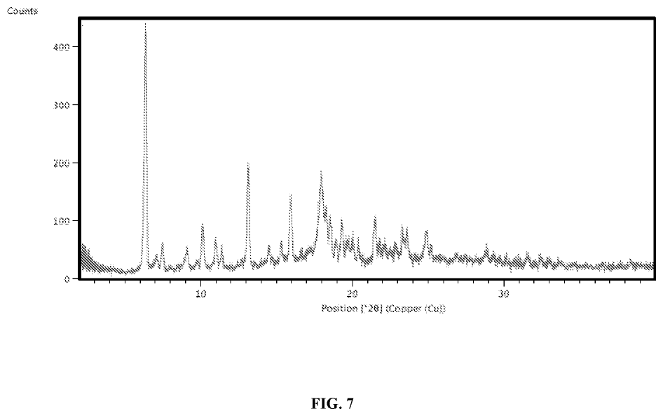

In some embodiments, the Form F crystal may be characterized by a powder X-ray diffraction pattern having powder X-ray diffraction peaks substantially as shown in Table 3. In some embodiments, the Form F crystal may be characterized by a powder X-ray diffraction pattern that may be substantially similar to the powder X-ray diffraction pattern of FIG. 7.

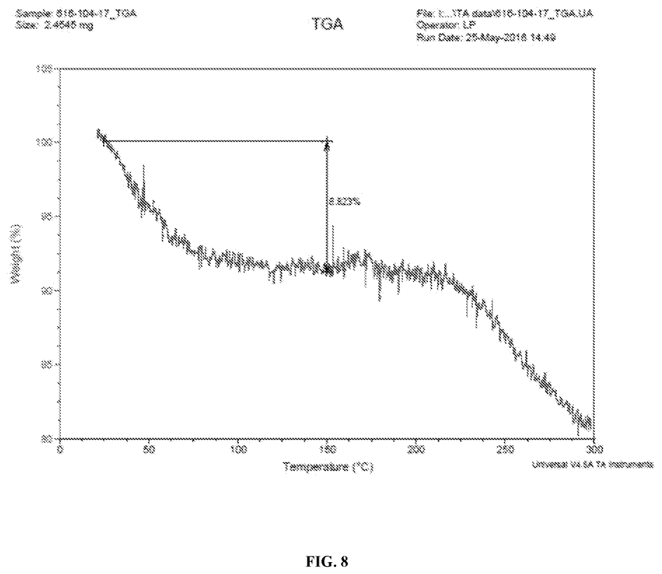

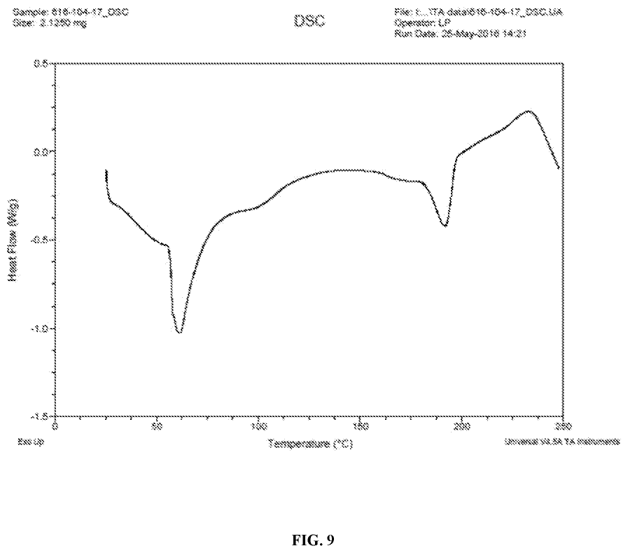

In some embodiments, the Form F crystal may be characterized by a thermal gravimetric analysis (TGA) curve substantially similar to the TGA in FIG. 8. In some embodiments, the Form F crystal may be characterized by a differential scanning calorimetry (DSC) measurement substantially similar to the DSC in FIG. 9. In some embodiments, the Form F crystal may be substantially pure.

TABLE 3

| No. | °20 |
|---|---|
| 1 | 2.1 |
| 2 | 6.4 |
| 3 | 7.1 |
| 4 | 7.5 |
| 5 | 9.1 |
| 6 | 10.1 |
| 7 | 11.0 |
| 8 | 11.4 |
| 9 | 13.2 |
| 10 | 14.5 |
| 11 | 15.3 |
| 12 | 15.9 |
| 13 | 18.0 |
| 14 | 18.6 |
| 15 | 18.9 |
| 16 | 19.3 |
| 17 | 20.0 |
| 18 | 20.4 |
| 19 | 21.5 |
| 20 | 23.6 |
| 21 | 24.9 |
| 22 | 28.8 |
| 23 | 31.6 |
| 24 | 32.4 |

The X-ray powder diffraction data of each of the above crystalline forms were obtained by using X-ray source as Cu, (Kα radiation, λ=1.54 Å).

Pharmaceutical dosage forms of crystals of 5-({[2-amino-3-(4-carbamoyl-2,6-dimethyl-phenyl)-propionyl]-[1-(4-phenyl-1H-imidazol-2-yl)-ethyl]-amino}-methyl)-2-methoxy-benzoic acid can be administered in several ways including, but not limited to, oral administration. Oral pharmaceutical compositions and dosage forms are exemplary dosage forms. Optionally, the oral dosage form is a solid dosage form, such as a tablet, a caplet, a hard gelatin capsule, a starch capsule, a hydroxypropyl methylcellulose (HPMC) capsule, or a soft elastic gelatin capsule. Liquid dosage forms may also be provided by the present invention, including such non-limiting examples as a suspension, a solution, syrup, or an emulsion. In another embodiment, the present invention includes the preparation of a medicament comprising a crystalline or polymorphic form of 5-({[2-amino-3-(4-carbamoyl-2,6-dimethyl-phenyl)-propionyl]-[1-(4-phenyl-1h-imidazol-2-yl)-ethyl]-amino}-methyl)-2-methoxy-benzoic acid. A Form E crystal of 5-({[2-amino-3-(4-carbamoyl-2,6-dimethyl-phenyl)-propionyl]-[1-(4-phenyl-1h-imidazol-2-yl)-ethyl]-amino}-methyl)-2-methoxy-benzoic acid can be administered by controlled- or delayed-release means.

Like the amounts and types of excipients, the amounts and specific type of active ingredient in a dosage form may differ depending on factors such as, but not limited to, the route by which it is to be administered to mammals. However, typical dosage forms of the invention comprise a Form D, E or F crystal of 5-({[2-amino-3-(4-carbamoyl-2,6-dimethyl-phenyl)-propionyl]-[1-(4-phenyl-1h-imidazol-2-yl)-ethyl]-amino}-methyl)-2-methoxy-benzoic acid, in an amount of from about 0.10 mg to about 1 g, from about 0.2 mg to about 500 mg, or from about 1 mg to about 250 mg. Non-limiting examples include 0.2 mg, 0.5 mg, 0.75 mg, 1.0 mg, 1.2 mg, 1.5 mg, 2 mg, 3 mg, 5 mg, 7 mg, 10 mg, 25 mg, 50 mg, 75 mg, 100 mg, 150 mg, 200 mg, 250 mg, and 500 mg dosages. In some embodiments, the dosage forms comprise 75 mg or 100 mg dosages. The dosages, however, may be varied depending upon the requirement of the patients, the severity of the condition being treated and the compound being employed. The use of either daily administration or post-periodic dosing may be employed.

The crystals of 5-({[2-amino-3-(4-carbamoyl-2,6-dimethyl-phenyl)-propionyl]-[1-(4-phenyl-1H-imidazol-2-yl)-ethyl]-amino}-methyl)-2-methoxy-benzoic acid of the present invention may also be used to prepare pharmaceutical dosage forms other than the oral dosage forms described above, such as topical dosage forms, parenteral dosage forms, transdermal dosage forms, and mucosal dosage forms. For example, such forms include creams, lotions, solutions, suspensions, emulsions, ointments, powders, patches, suppositories, and the like.

The crystals of 5-({[2-amino-3-(4-carbamoyl-2,6-dimethyl-phenyl)-propionyl]-[1-(4-phenyl-1h-imidazol-2-yl)-ethyl]-amino}-methyl)-2-methoxy-benzoic acid of the present invention can be characterized by the TGA or DSC data, or by any one, any two, any three, any four, any five, any six, any seven, any eight, any nine, or any ten PXRD 2-theta angle peaks, or by any combination of the data acquired from the analytical techniques described above which distinctly identify the particular crystal.

In an embodiment, a pharmaceutical composition of this invention also may include combinations of the different crystalline forms of eluxadoline described herein, amorphous eluxadoline or crystalline Forms a and R as described in U.S. Publication No. 2005/02033143. A single pharmaceutical composition may include two, three, four, or more than four different crystalline forms of eluxadoline. For example, a pharmaceutical composition may be composed of Forms D and E; D and F; D and amorphous eluxadoline; D and α; D and β; E and F; E and amorphous eluxadoline; E and α; E and β; F and amorphous eluxadoline; F and α; or F and β.

The present invention is also directed to methods of isolating and preparing the crystal forms D, E and F of 5-({[2-amino-3-(4-carbamoyl-2,6-dimethyl-phenyl)-propionyl]-[1-(4-phenyl-1H-imidazol-2-yl)-ethyl]-amino}-methyl)-2-methoxy-benzoic acid.

In some embodiments, the methods comprise first preparing Form A crystal of eluxadoline, which involve the steps of combining a strong ionizable acid eluxadoline to prepare a salt of eluxadoline; and washing said salt of eluxadoline with an inorganic base to obtain eluxadoline. In another embodiment, Form A crystals may be made in the process described in U.S. Pub. No. 2005/02033143, the contents of which are incorporated herein in their entirety. In some embodiments, the invention may further comprise the step of washing said eluxadoline with water. The inorganic base may be selected from sodium hydroxide, potassium hydroxide, sodium carbonate, sodium acetate, sodium phosphate. In some embodiments, the inorganic base is sodium hydroxide. The ionizable acid may be selected from hydrochloric acid, trifluoroacetic acid, sulphuric acid, formic acid, and phosphoric acid. In some embodiments, said ionizable acid is hydrochloric acid. In one embodiment, a method of preparing eluxadoline comprises the steps of: combining hydrochloric acid with eluxadoline to prepare the hydrochloride salt of eluxadoline; washing said salt of eluxadoline with sodium hydroxide; and washing said eluxadoline with water. The resulting eluxadoline is then added to dichloromethane and heated, the slurry stored at room temperature, the residue isolated and dried to prepare Form A crystal. In some embodiments, Form A is suspended in water and stirred for a period of time, subsequently solids isolated, rinsed and dried to prepare Form D crystal. In some embodiments, Form D is stored in the presence of a drying agent to prepare Form E crystal. In further embodiments, Form A is suspended in a mixture of 2-propoanol and water, Forms D and E added to the mixture, subsequently solids isolated and allowed to dry to prepare Form F crystal.

In one embodiment, the crystalline forms of this invention have improved stability than the amorphous form.

The crystals of the present invention were analyzed using the following methods.

### Differential Scanning Calorimetry

Differential scanning calorimetry was performed with a TA Discovery series DSC using a few milligrams of material in a Tzero aluminum pan sealed with a Tzero hermetic lid containing two pin holes. Unless otherwise specified, samples were scanned at 10° C. per minute under 50 mL per minute of nitrogen flow.

One of skill in the art will however, note that in DSC measurement there is a certain degree of variability in actual measured onset and peak temperatures, depending on rate of heating, crystal shape and purity, sample preparation and other measurement parameters.

### Powder X-Ray Diffraction

X-ray powder diffraction data was collected under ambient conditions by placing samples on a zero background holder with a 0.1 mm indent and generated using a Rigaku Miniflex 600 diffractometer with Cu K alpha (1.5406 Angstrom) radiation at a scan rate of 2 to 40° 2θ at 2° per min at 40 kV and 15 mA.

One of ordinary skill in the art will appreciate that a powder X-ray diffraction pattern may be obtained with a measurement error that is dependent upon the measurement conditions employed. In particular, it is generally known that intensities in a X-ray powder diffraction pattern may fluctuate depending upon measurement conditions employed. It should be further understood that relative intensities may also vary depending upon experimental conditions and, accordingly, the exact order of intensity should not be taken into account. Accordingly, the relative intensity of peaks in a diffractogram is not necessarily a limitation of the PXRD pattern because peak intensity can vary from sample to sample, e.g., due to crystalline impurities.

Additionally, a measurement error of diffraction angle for a conventional powder X-ray powder diffraction pattern is typically about 5% or less, and such degree of measurement error should be taken into account as pertaining to the aforementioned diffraction angles. Further, the angles of each peak can vary by about +/−0.1 degrees, or by about +/−0.05. The entire pattern or most of the pattern peaks may also shift by about +/−0.1 degrees to about +/−0.2 degrees due to differences in calibration, settings, and other variations from instrument to instrument. All reported PXRD peaks in the Figures, Examples, and elsewhere herein are reported with an error of about 0.2 degrees 2-theta. Unless otherwise noted, all diffractograms are obtained at about room temperature (about 24 degrees C. to about 25 degrees C.). It is to be understood that the crystal structures of the instant invention are not limited to the crystal structures that provide X-ray diffraction patterns completely identical to the X-ray powder diffraction patterns depicted in the accompanying Figures disclosed herein. Any crystal structures that provide powder X-ray diffraction patterns substantially identical to those disclosed in the accompanying Figures fall within the scope of the present invention. The ability to ascertain substantial identities of X-ray powder diffraction patterns is within the purview of one of ordinary skill in the art.

### Thermal Gravimetric Analysis

Thermogravimetric analysis data was collected with a TA Discovery series TGA. A few milligrams of material were analyzed in an aluminum sample pan. The data was collected from room temperature to 300° C. with a 10° C. per min scan rate.

One of skill in the art will however, note that in TGA measurement there is a certain degree of variability in the measured onset and peak temperatures, depending on rate of heating, crystal shape and purity, sample preparation and other measurement parameters.

The following specific examples illustrate the present invention in more detail. They are, however, not intended to limit its scope in any manner.

### EXAMPLES

### Example 1: Preparation of the Form D Crystal of 5-({[2-Amino-3-(4-carbamoyl-2,6-dimethyl-phenyl)-propionyl]-[1-(4-phenyl-1H-imidazol-2-yl)-ethyl]-amino}-methyl)-2-methoxy-benzoic acid

A 1 L three-necked round-bottomed flask equipped with a mechanical stirrer, addition funnel and a thermocouple was charged without agitation. 34.2 g of 5-({[2-tert-butoxycarbonylamino-3-(4-carbamoyl-2,6-dimethyl-phenyl)-propionyl]-[1-(4-phenyl-1H-imidazol-2-yl)-ethyl]-amino}-methyl)-2-methoxy-benzoic acid (see Example 9 of US 2005/0203143), 340 mL of acetone, and 17 mL of 204 mM concentrated HCl were combined in the flask. The stirring was started and the resulting slurry formed a clear solution. This solution was heated to 45° C. under vigorous stirring and aged at this temperature for a period of two hours. After the completion, the reaction mass was cooled to ambient temperature and the supernatant was removed by suction. The vessel along with the residue was rinsed with 20 ml of acetone and then removed as previously. 170 ml of water was added and the reaction mass and was aged under stirring until a homogeneous solution resulted. This solution was then added over a period of ˜½ hr to a solution of 90 ml of 1N NaOH and water. The pH was adjusted to 6.5-7.0 accordingly. The resulting slurry was aged for about 2 hrs at ambient temperature, cooled to 10-15° C., aged at that temperature for about 1 hr, and then filtered. The solid was washed with 10 ml water, air-dried for a period of 4 to 5 hrs, and then placed in a vacuum oven at 50-55° C. until the water content was less than 3%. The resulting zwitterion of 5-({[2-tert-butoxycarbonylamino-3-(4-carbamoyl-2,6-dimethyl-phenyl)-propionyl]-[1-(4-phenyl-1H-imidazol-2-yl)-ethyl]-amino}-methyl)-2-methoxy-benzoic acid was added to 3 ml dichloromethane and heated at 50° C. for 2 days. The slurry was stored at room temperature for 3 days. The residue was isolated and dried for 4 days at 40° C. to prepare Form A crystal.

Form D was prepared by suspending 3 grams of Form A in 12 mL of water in a 20 mL vial. The contents of the vial were stirred for 9 days at 25° C. Solids were isolated by vacuum filtration, rinsed with water, and air dried. Form D is a tetrahydrate form of eluxadoline. Characterization data of Form D including XRPD (FIG. 1), an XRPD peak list (Table 1), DSC (FIG. 2) and TGA (FIG. 3) are shown herein.

### Example 2: Preparation of the Form E Crystal

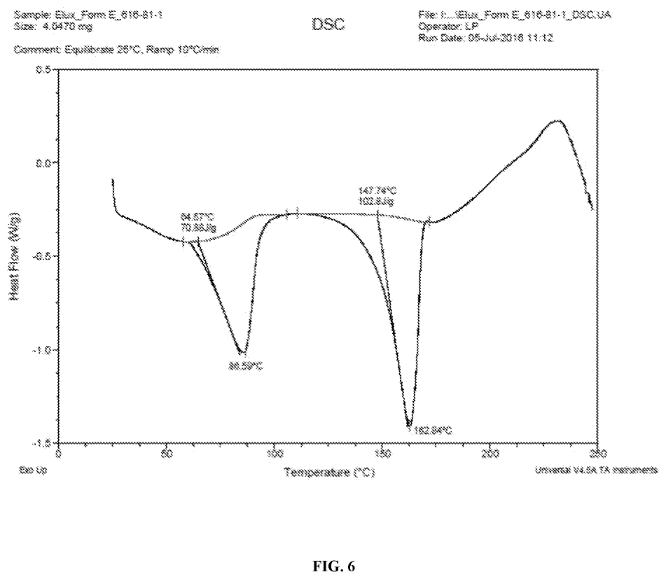

Form E was prepared by storing 1 gram of Form D in a desiccator containing Dririte as the drying agent for 3 days at ambient temperature. Form E is a partially dehydrated form of eluxadoline Form D. Characterization data of Form E including XRPD (FIG. 4), an XRPD peak list (Table 2), DSC (FIG. 5) and TGA (FIG. 6) are shown herein.

### Example 3: Preparation of the Form F Crystal

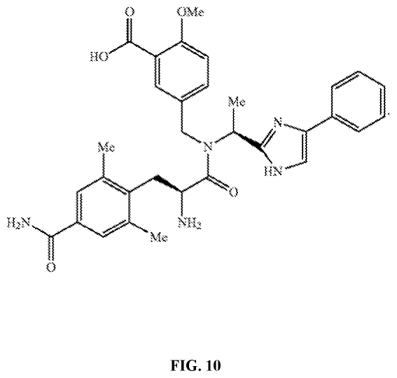

Form F was prepared by suspending 39 mg of Form A in 1 mL of 70% 2-propanol/30% water in a 4 mL vial. The contents of the vial stirred for 1 day at 25° C. Then 37 mg of Form D and 33 mg of Form E were added and vial continued to stir at 25° C. for 4 weeks. Solids of Form F were isolated by centrifuging, discarding the supernatant, and allowing solids to air dry. Form F is a solvated form of eluxadoline. Characterization data of Form F including XRPD (FIG. 7), an XRPD peak list (Table 3), DSC (FIG. 8), TGA (FIG. 9), and solution NMR (FIG. 10) are shown herein.

Although the invention has been described with respect to various embodiments, it should be realized this invention is also capable of a wide variety of further and other embodiments within the spirit and scope of the appended claims

What is claimed:

1. A pharmaceutical composition comprising a Form D crystal of 5-({[2-amino-3-(4-carbamoyl-2,6-dimethyl-phenyl)-propionyl]-[1-(4-phenyl-1H-imidazol-2-yl)-ethyl]-amino}-methyl)-2-methoxy-benzoic acid, wherein the Form D crystal is characterized by a powder X-ray diffraction pattern having at least a minimum corresponding number of powder X-ray diffraction peaks selected from the group consisting of powder X-ray diffraction peaks at about 8.0±0.2, 8.6±0.2, 8.9±0.2, 10.1±0.2, 11.0±0.2, 13.4±0.2, 14.7±0.2, 15.8±0.2, 17.7±0.2, 18.6±0.2, 20.2±0.2, 21.3±0.2, 22.1±0.2, 23.2±0.2, 24.2±0.2, 25.6±0.2, 26.9±0.2, 28.2±0.2, 29.2±0.2, 30.0±0.2, 31.0±0.2, 32.1±0.2, 33.4±0.2, 34.5±0.2, 36.7±0.2, and 38.2±0.2 degrees 2-theta, wherein said minimum corresponding number is three.

2. The pharmaceutical composition of claim 1, wherein the Form D crystal is characterized by a powder X-ray diffraction pattern having any three or more powder X-ray diffraction peaks selected from the group consisting of powder X-ray diffraction peaks at about 8.0±0.2, 8.6±0.2, 8.9±0.2, 10.1±0.2, 11.0±0.2, 13.4±0.2, 14.7±0.2, 15.8±0.2, 17.7±0.2, 18.6±0.2, 20.2±0.2, 21.3±0.2, 22.1±0.2, 23.2±0.2, 24.2±0.2, 25.6±0.2, 26.9±0.2, 28.2±0.2, 29.2±0.2, 30.0±0.2, 31.0±0.2, 32.1±0.2, 33.4±0.2, 34.5±0.2, 36.7±0.2, and 38.2±0.2 degrees 2-theta.

3. The pharmaceutical composition of claim 1, wherein the Form D crystal is characterized by a powder X-ray diffraction pattern having powder X-ray diffraction peaks at about 17.7±0.2. 20.2±0.2 and 26.9±0.2 degrees 2-theta.

4. The pharmaceutical composition of claim 1, wherein the Form D crystal is characterized by a powder X-ray diffraction pattern having powder X-ray diffraction peaks at about 8.9±0.2, 17.7±0.2. 20.2±0.2 and 26.9±0.2 degrees 2-theta.

5. The pharmaceutical composition of claim 1, wherein the Form D crystal is characterized by a powder X-ray diffraction pattern having powder X-ray diffraction peaks at about 8.6±0.2, 8.9±0.2, 17.7±0.2. 20.2±0.2 and 26.9±0.2 degrees 2-theta.

6. The pharmaceutical composition of claim 1, wherein said minimum corresponding number is four.

7. The pharmaceutical composition of claim 1, wherein the Form D crystal is characterized by a powder X-ray diffraction pattern having powder X-ray diffraction peaks substantially similar to the powder X ray diffraction peaks of FIG. 1.

8. The pharmaceutical composition of claim 1, wherein the Form D crystal is characterized by a differential scanning calorimetry (DSC) measurement substantially similar to the DSC in FIG. 3.

9. The pharmaceutical composition of claim 1, wherein the Form D crystal is characterized by a thermal gravimetric analysis (TGA) substantially similar to the TGA in FIG. 2.

10. The pharmaceutical composition of claim 1, in a dosage form suitable for oral administration.

11. The pharmaceutical composition of claim 10, wherein the dosage form is a solid.

12. The pharmaceutical composition of claim 10, wherein the dosage form is selected from the group consisting of a tablet, a caplet, a hard gelatin capsule, a starch capsule, a hydroxypropyl methylcellulose (HPMC) capsule, and a soft elastic gelatin capsule.

13. The pharmaceutical composition of claim 10, wherein the dosage form as administered is a liquid.

14. The pharmaceutical composition of claim 10, wherein the dosage form as administered is selected from the group consisting of a suspension, a solution, a syrup, and an emulsion.

15. The pharmaceutical composition of claim 10, wherein the dosage form is a tablet.

16. A method of treating an opioid receptor disorder in a mammal comprising administering to the mammal an effective amount of the pharmaceutical composition of claim 1.

17. The method of claim 16, wherein the opioid receptor disorder is selected from the group consisting of irritable bowel syndrome, pain and a combination of both.

18. The method of claim 16, wherein the opioid receptor disorder is irritable bowel syndrome.

19. The method of claim 16, wherein the opioid receptor disorder is pain.

20. A pharmaceutical composition comprising a Form E crystal of 5-({[2-amino-3-(4-carbamoyl-2,6-dimethyl-phenyl)-propionyl]-[1-(4-phenyl-1H-imidazol-2-yl)-ethyl]-amino}-methyl)-2-methoxy-benzoic acid, wherein the Form E crystal is characterized by a powder X-ray diffraction pattern having at least a minimum corresponding number of powder X-ray diffraction peaks selected from the group consisting of powder X-ray diffraction peaks at about 2.1±0.2, 8.6±0.2, 10.3±0.2, 11.2±0.2, 11.8±0.2, 13.5±0.2, 15.3±0.2, 15.8±0.2, 17.4±0.2, 18.3±0.2, 19.4±0.2, 20.1±0.2, 21.6±0.2, 23.1±0.2, 24.5±0.2, 25.7±0.2, 27.8±0.2, 28.8±0.2, 30.2±0.2, 32.5±0.2, 33.4±0.2, 35.2±0.2 and 38.4±0.2.±0.2 degrees 2-theta, wherein said minimum corresponding number is three.

21. The pharmaceutical composition of claim 20, wherein the Form E crystal is characterized by a powder X-ray diffraction pattern having any three or more powder X-ray diffraction peaks selected from the group consisting of powder X-ray diffraction peaks at about 2.1±0.2, 8.6±0.2, 10.3±0.2, 11.2±0.2, 11.8±0.2, 13.5±0.2, 15.3±0.2, 15.8±0.2, 17.4±0.2, 18.3±0.2, 19.4±0.2, 20.1±0.2, 21.6±0.2, 23.1±0.2, 24.5±0.2, 25.7±0.2, 27.8±0.2, 28.8±0.2, 30.2±0.2, 32.5±0.2, 33.4±0.2, 35.2±0.2 and 38.4±0.2.±0.2 degrees 2-theta.

22. The pharmaceutical composition of claim 20, wherein the Form E crystal is characterized by a powder X-ray diffraction pattern having powder X-ray diffraction peaks at about 8.6±0.2. 17.4±0.2 and 21.6±0.2 degrees 2-theta.

23. The pharmaceutical composition of claim 20, wherein the Form E crystal is characterized by a powder X-ray diffraction pattern having powder X-ray diffraction peaks at about 8.6±0.2, 11.8±0.2. 17.4±0.2 and 21.6±0.2 degrees 2-theta.

24. The pharmaceutical composition of claim 20, wherein the Form E crystal is characterized by a powder X-ray diffraction pattern having powder X-ray diffraction peaks at about 8.6±0.2, 11.8±0.2, 17.4±0.2. 21.6±0.2 and 27.8±0.2 degrees 2-theta.

25. The pharmaceutical composition of claim 20, wherein said minimum corresponding number is four.

26. The pharmaceutical composition of claim 20, wherein the Form E crystal is characterized by a powder X-ray diffraction pattern having powder X-ray diffraction peaks substantially similar to the powder X ray diffraction peaks of FIG. 4.

27. The pharmaceutical composition of claim 20, wherein the Form E crystal is characterized by a differential scanning calorimetry (DSC) measurement substantially similar to the DSC in FIG. 6.

28. The pharmaceutical composition of claim 20, wherein the Form E crystal is characterized by a thermal gravimetric analysis (TGA) substantially similar to the TGA in FIG. 5.

29. The pharmaceutical composition of claim 20, in a dosage form suitable for oral administration.

30. The pharmaceutical composition of claim 29, wherein the dosage form is a solid.

31. The pharmaceutical composition of claim 29, wherein the dosage form is selected from the group consisting of a tablet, a caplet, a hard gelatin capsule, a starch capsule, a hydroxypropyl methylcellulose (HPMC) capsule, and a soft elastic gelatin capsule.

32. The pharmaceutical composition of claim 29, wherein the dosage form as administered is a liquid.

33. The pharmaceutical composition of claim 29, wherein the dosage form as administered is selected from the group consisting of a suspension, a solution, a syrup, and an emulsion.

34. The pharmaceutical composition of claim 29, wherein the dosage form is a tablet.

35. A method of treating an opioid receptor disorder in a mammal comprising administering to the mammal an effective amount of the pharmaceutical composition of claim 20.

36. The method of claim 35, wherein the opioid receptor disorder is selected from the group consisting of irritable bowel syndrome, pain and a combination of both.

37. The method of claim 35, wherein the opioid receptor disorder is irritable bowel syndrome.

38. The method of claim 35, wherein the opioid receptor disorder is pain.

39. A pharmaceutical composition comprising a Form F crystal of 5-({[2-amino-3-(4-carbamoyl-2,6-dimethyl-phenyl)-propionyl]-[1-(4-phenyl-1H-imidazol-2-yl)-ethyl]-amino}-methyl)-2-methoxy-benzoic acid, wherein the Form F crystal is characterized by a powder X-ray diffraction pattern having at least a minimum corresponding number of powder X-ray diffraction peaks selected from the group consisting of powder X-ray diffraction peaks at about 2.1±0.2, 6.4±0.2, 7.1±0.2, 7.5±0.2, 9.1±0.2, 10.1±0.2, 11.0±0.2, 11.4±0.2, 13.2±0.2, 14.5±0.2, 15.9±0.2, 18.0±0.2, 18.6±0.2, 18.9±0.2, 19.3±0.2, 20.0±0.2, 20.4±0.2, 21.5±0.2, 23.6±0.2, 24.9±0.2, 28.8±0.2. 31.6±0.2 and 32.4±0.2 degrees 2-theta, wherein said minimum corresponding number is three.

40. The pharmaceutical composition of claim 39, wherein the Form F crystal is characterized by a powder X-ray diffraction pattern having any three or more powder X-ray diffraction peaks selected from the group consisting of powder X-ray diffraction peaks at about 2.1±0.2, 6.4±0.2, 7.1±0.2, 7.5±0.2, 9.1±0.2, 10.1±0.2, 11.0±0.2, 11.4±0.2, 13.2±0.2, 14.5±0.2, 15.9±0.2, 18.0±0.2, 18.6±0.2, 18.9±0.2, 19.3±0.2, 20.0±0.2, 20.4±0.2, 21.5±0.2, 23.6±0.2, 24.9±0.2, 28.8±0.2. 31.6±0.2 and 32.4±0.2 degrees 2-theta.

41. The pharmaceutical composition of claim 39, wherein the Form F crystal is characterized by a powder X-ray diffraction pattern having powder X-ray diffraction peaks at about 6.4±0.2. 13.2±0.2 and 18.0±0.2 degrees 2-theta.

42. The pharmaceutical composition of claim 39, wherein the Form F crystal is characterized by a powder X-ray diffraction pattern having powder X-ray diffraction peaks at about 6.4±0.2. 13.2±0.2, 15.9±0.2 and 18.0±0.2 degrees 2-theta.

43. The pharmaceutical composition of claim 39, wherein the Form F crystal is characterized by a powder X-ray diffraction pattern having powder X-ray diffraction peaks at about 6.4±0.2. 10.1±0.2, 13.2±0.2, 15.9±0.2 and 18.0±0.2 degrees 2-theta.

44. The pharmaceutical composition of claim 39, wherein said minimum corresponding number is four.

45. The pharmaceutical composition of claim 39, wherein the Form F crystal is characterized by a powder X-ray diffraction pattern having powder X-ray diffraction peaks substantially similar to the powder X ray diffraction peaks of FIG. 7.

46. The pharmaceutical composition of claim 39, wherein the Form F crystal is characterized by a differential scanning calorimetry (DSC) measurement substantially similar to the DSC in FIG. 9.

47. The pharmaceutical composition of claim 39, wherein the Form F crystal is characterized by a thermal gravimetric analysis (TGA) substantially similar to the TGA in FIG. 8.

48. The pharmaceutical composition of claim 39, in a dosage form suitable for oral administration.

49. The pharmaceutical composition of claim 39, wherein the dosage form is a solid.

50. The pharmaceutical composition of claim 29, wherein the dosage form is selected from the group consisting of a tablet, a caplet, a hard gelatin capsule, a starch capsule, a hydroxypropyl methylcellulose (HPMC) capsule, and a soft elastic gelatin capsule.

51. The pharmaceutical composition of claim 50, wherein the dosage form as administered is a liquid.

52. The pharmaceutical composition of claim 50, wherein the dosage form as administered is selected from the group consisting of a suspension, a solution, a syrup, and an emulsion.

53. The pharmaceutical composition of claim 50, wherein the dosage form is a tablet.

54. A method of treating an opioid receptor disorder in a mammal comprising administering to the mammal an effective amount of the pharmaceutical composition of claim 39.

55. The method of claim 54, wherein the opioid receptor disorder is selected from the group consisting of irritable bowel syndrome, pain and a combination of both.

56. The method of claim 54, wherein the opioid receptor disorder is irritable bowel syndrome.

57. The method of claim 54, wherein the opioid receptor disorder is pain.
